(19) United States
(12) Patent Application Publication
(10) Pub. No.: US 2015/0297856 A1
(43) Pub. Date: Oct. 22, 2015

(54) AIRWAY DEVICE WITH INTEGRATED BREATH SAMPLING

(71) Applicant: ORIDION MEDICAL 1987 LTD., Jerusalem (IL)

(72) Inventor: Seamus Maguire, Athlone (IE)

(73) Assignee: ORIDION MEDICAL 1987 LTD., Jerusalem (IL)

(21) Appl. No.: 14/258,542

(22) Filed: Apr. 22, 2014

(51) Int. Cl. A61M 16/08; A61M 16/01; A61M 16/00; A61B 5/097; A61M 16/04

(52) U.S. Cl. CPC A61M 16/085; A61B 5/097; A61M 16/0465; A61M 16/0057; A61M 16/01; A61M 2210/1035

(57) ABSTRACT

An airway device having a shaft including an airway channel, the airway channel having an outer and an inner wall; a sampling channel; and a luer connector mounted on the outer wall such that respiratory gasses flowing in the sampling channel are allowed to exit through the luer connector to a sampling tube connected to the luer connector.

### TECHNICAL FIELD

The present disclosure relates generally to the field of airway devices and breath sampling.

### BACKGROUND

When a patient is mechanically ventilated a breathing circuit is generated between airway devices, such as endotracheal tubes, endobroncheal tubes and tracheostomy tubes, and the ventilator machine. The circuit must be a closed conduit in order to ensure effective ventilation of the patient's lungs.

In order to monitor carbon dioxide (CO₂) level of the ventilated patient a sampling line must be coupled to the circuit, preferably close to the patient end of the circuit. This is typically done by connecting the sampling line to the breath circuit using a double sided connector.

### SUMMARY

Aspects of the disclosure, in some embodiments thereof, relate to airway devices with integrated breath sampling. The airway devices disclosed herein may include luer connectors mounted on the airway device and allowing samples of gas flowing in the airway device to exit through the luer connector directly to a sampling tube connected to thereto.

During side stream monitoring of a patient's CO₂ levels, a sampling line must be connected to the breathing circuit. This is typically done using double sided connectors. The airway device disclosed herein, integrates CO₂ side stream sampling within the airway device itself. Advantageously, this eliminates the need for double ended connectors. In effect, a decrease in the overall weight of the breathing circuit is achieved thereby improving patient comfort. Moreover, sampling accuracy may be refined due to avoidance of leakages and due to a reduced volume of the breathing circuit. Often multiple connectors connecting additional medical equipment, such as suction catheters and the like, compete for the same area of connection. This typically results in distancing the sampling line from the patient consequently negatively impacting the response time of the CO₂. Favorably, the airway device, disclosed herein, devoid double sided connectors, takes up much less space and thus facilitates connection of the sampling tube in close proximity to the patient while minimizing interference with additional medical equipment likewise connected to the airway device.

According to some embodiments, there is provided an airway device including a shaft having an airway channel configured to allow flow of respiratory gasses, the airway channel having an outer and an inner wall, a sampling channel, and a luer connector mounted on the outer wall such that respiratory gasses are allowed to flow from the airway channel through the sampling channel and the luer connector to a sampling tube connected to the luer connector.

According to some embodiments, the sampling channel extends through the inner and outer walls of the airway channel. According to some embodiments, the sampling channel is sloped relative to the airway channel. According to some embodiments, the sampling channel is embedded in said outer wall of said airway channel. According to some embodiments, the sampling channel extends along at least a part of the airway channel.

According to some embodiments, the airway device further includes a notch configured to allow gas to flow into said sampling channel. According to some embodiments, the notch is located in an outer surface of the shaft thereby allowing breath to flow from the patient's airway to the sampling channel. According to some embodiments, the notch is located in an inner surface of the shaft, thereby allowing breath to flow from the airway channel to the sampling channel. According to some embodiments, the notch is positioned at a distal end of the shaft inside a patient's body.

According to some embodiments, the luer connector is mounted on a proximal end of the shaft externally to a patient's body.

According to some embodiments, the luer connector includes a sampling probe configured to sample gas from an inner diameter of the airway channel. According to some embodiments, the sampling probe is spring loaded. According to some embodiments, the sampling probe is normally retracted within the luer connector. According to some embodiments, the sampling probe enters into the airway channel when the sampling tube is connected to the luer connector.

According to some embodiments, the luer connector further includes a seal configured to prevent gasses flowing in the airway channel to exit through the luer connector when no sampling tube is connected thereto.

According to some embodiments, the airway device includes an endotracheal tube, an endobroncheal tube or a tracheostomy tube.

According to some embodiments, the airway device is configured to be connected to a ventilation, machine or an anesthetics machine.

According to some embodiments, there is provided an airway device including a shaft having an airway channel configured to allow flow of respiratory gasses, a sampling channel extending along at least a part of the airway channel and a sampling tube coextensive with and permanently attached to the sampling channel.

According to some embodiments, the airway device further includes a notch configured to allow gas to flow into said sampling channel. According to some embodiments, the notch is located in an outer surface of the shaft thereby allowing breath to flow from the patient's airway to the sampling channel. According to some embodiments, the notch is located in an inner surface of the shaft, thereby allowing breath to flow from the airway channel to the sampling channel. According to some embodiments, the notch is positioned at a distal end of the shaft inside a patient's body.

According to some embodiments, there is provided an airway system including a breath sampling tube; and an airway device. According to some embodiments, the airway device includes a shaft having an airway channel configured to allow gas flow therein, the airway channel having an outer and an inner wall. According to some embodiments, the airway device further includes a sampling channel and a luer connector mounted on the outer wall such that part of the gas flowing in the sampling channel is allowed to exit through the luer connector to the sampling tube connected to the luer connector.

According to some embodiments, the airway system further includes a ventilation machine and/or an anesthetics machine.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

### BRIEF DESCRIPTION OF THE DRAWINGS

Some embodiments of the disclosure are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the teachings of the disclosure. For the sake of clarity, some objects depicted in the figures are not to scale.

FIG. 1A schematically illustrates an airway device with integrated breath sampling, according to some embodiments;

FIG. 1B schematically illustrates an airway device with integrated breath sampling connected to a sampling tube, according to some embodiments;

FIG. 2A schematically illustrates an airway device with a sampling probe, according to some embodiments;

FIG. 2B schematically illustrates an airway device with a sampling probe connected to a sampling tube, according to some embodiments;

FIG. 3A schematically illustrates an airway device with a sampling lumen and an notch in an outer surface thereof, according to some embodiments;

FIG. 3B schematically illustrates an airway device with a sampling lumen and an notch in an inner surface thereof, according to some embodiments;

FIG. 4 schematically illustrates an airway device with a sampling lumen coextensive with as sampling tube, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present disclosure relates generally to airway devices with integrated breath sampling. The airway device includes luer connectors mounted thereon allowing connection of breath sampling tubes directly to the airway device thereby eliminating the need for the quite cumbersome double sided connectors typically used to interconnect sampling tubes (as well as other consumables) to the airway device.

According to some embodiments, there is provided an airway device having a shaft including an airway channel configured to allow gas flow therein, the airway channel having an outer and an inner wall.

As used herein, the term “airway device” may refer to tubes used for ventilating patients such as but not limited to endotracheal tubes, endobroncheal tubes and tracheostomy tubes configured to be connected to a ventilation and/or an anesthetics machine. According to some embodiments, the term “shaft”, as used herein may refer to part of the airway device utilized for exchanging the gas flowing to and from the patient. According to some embodiments, the distal end of the shaft is configured to be placed inside the patient (for example in the trachea of the patient). According to some embodiments, the proximal end of the shaft is located externally to the patient.

As used herein, the terms “breath sampling tube”, “sampling line” and “breath sampling line” may refer to any type of tubing(s) or any part of tubing system adapted to allow the flow of sampled breath, for example, to a breath analyzer, such as a capnograph. The sampling line may include tubes of various diameters, adaptors, connectors, valves, drying elements (such as filters, traps, trying tubes, such as Nafion® and the like).

As referred to herein, the terms “patient” and “subject” may interchangeably be used and may relate to a subject being connected to an airway device.

According to some embodiments, the terms “gas” and “respiratory gas” may be interchangeably used and may refer to the gasses flowing in the respiratory circuit (between the patient and the ventilation machine. According to some embodiments, the gas may be exhaled breath. According to some embodiments, the gas may be the respiratory gas supplied by the ventilation machine.

According to some embodiments, the airway device has a sampling channel. According to some embodiments, the term “sampling channel” may refer a channel extending through the inner and outer wall of the shaft.

According to some embodiments, the sampling channel may be essentially perpendicular to the airway channel. According to some embodiments, the sampling channel is sloped relative to said airway channel. This augments the entrance of gasses flowing from the patient (exhaled breath) as compared to gases supplied to the patient by the ventilation machine.

According to some embodiments, the sampling channel may be a lumen formed in and along at least part of the shaft. According to some embodiments, the sampling lumen is an integral part of the shaft. According to some embodiments, the sampling lumen terminates distally (in direction of the medical device) to the airway channel. According to some embodiments the sampling lumen terminates proximally (deeper inside the patient) to the airway channel. According to some embodiments, the sampling lumen forms a double lumen structure with the airway channel. According to some embodiments, the sampling lumen is closed off at its terminal end (inside the patient) thereof.

According to some embodiments, the shaft includes a notch configured to allow gas to flow into the sampling lumen. As used herein, the terms “notch” and “cut” may be used interchangeably and may refer to any opening allowing respiratory gases to enter the sampling lumen. It is understood by one of ordinary skill in the art, that this configuration allows sampling inside the patient's body.

According to some embodiments, the notch is located in an external surface of said shaft. In effect, respiratory gases (exhaled breath) may enter the sampling lumen directly from the patient. According to some embodiments, the notch is located in an internal surface of the shaft. In effect, exhaled breath may enter the sampling lumen from the airway channel. According to some embodiments, the notch is located inside the airway channel proximally to the distal end of the shaft.

According to some embodiments, the notch is positioned on a distal end of the shaft, inside a patient's body.

According to some embodiments, the sampling channel is co-extensive with a sampling tube. According to some embodiments, the sampling tube may be permanently attached to the airway device. According to some embodiments, the sampling tube may be formed as an integral part of the airway device, thereby reducing cost of manufacturing. Advantageously, the permanently connected sampling tube may enable to plug in the airway device straight to the breath analyzer without requiring further interconnections. Such configuration is particularly suitable for airway devices used in routine and short procedures in which the relatively short life time of the sampling tube does not shorten the overall lifetime of the airway device.

According to some embodiments the airway device includes a luer connector mounted on the outer wall of the shaft such that gas flowing in the sampling channel is allowed to exit to a sampling tube connected to the luer connector.

According to some embodiments, luer connector is mounted on a distal end of the shaft externally to the patient's body.

As used herein the term “mounted on” may refer to the luer connector being embedded in, molded on, integrally formed with, contiguously formed with, or otherwise attached to the shaft. Each possibility is a separate embodiment. According to some embodiments, the luer connector may be mounted on a distal end of the shaft external to a patient's body. According to some embodiments, the luer connector may be mounted on the shaft in close proximity to the patient. As used herein the term “close proximity” may refer to 50, 30, 20, 15, 10, 5, 1, 0.5 cm or less from the patient. Each possibility is a separate embodiment.

As used herein, the terms “proximal” and “proximal end” may refer to the part of the tube closest to the medical device, such as a ventilation machine. The length of the proximal end may for example be 0.5, 1, 2, 3, 4, 5, 10 cm or more. Each possibility is a separate embodiment.

As used herein, the terms “distal” and “distal end” may refer to the part of the tube closest to the subject. The length of the distal end may for example be 0.5, 1, 2, 3, 4, 5, 10 cm or more. Each possibility is a separate embodiment.

As used herein, the term “certain distance” may refer to a distance larger than 10 cm, for example larger than 20 cm, 30 cm, 40 cm or 50 cm, 70 cm. Each possibility is a separate embodiment.

According to some embodiments, the luer connector may include a sampling probe configured to sample exhaled breath from an inner diameter of the airway channel. It is understood to one of ordinary skill in the art that sampling breath from an inner diameter of the airway channel ensures obtaining optimal CO₂ readings with minimal disturbances to the airflow. According to some embodiments, the term “sampling probe” may refer to a tube or other suitable element configured to sample breath from an inner diameter of the airway channel. According to some embodiments, the sampling probe may be spring loaded. This may facilitate the sampling probe to be normally retracted within the luer connector and to enter into the airway channel only when a sampling tube is connected to the luer connector. Thus the sampling probe disclosed herein, facilitates breath sampling from an inner diameter of the airway channel without hampering passage of instruments and/or devices therethrough.

According to some embodiments, the sampling probe is deposited within, molded on, integrally formed with, contiguously formed with, or otherwise attached within the luer connector. Each possibility is a separate embodiment.

According to some embodiments, the luer connector includes a seal configured to prevent gasses flowing in the airway channel to exit through the luer connector when no sampling tube is connected thereto. It is understood to one of ordinary skill in the art that the seal may be made of any material impermeable to the gasses flowing in the airway channel. According to some embodiments, the seal is configured to seal of the sampling channel proximately to its opening in the inner wall. Alternatively, the seal is configured to seal of the sampling channel proximately to the opening in the outer wall. According to some embodiments, the seal is detached, moved aside or otherwise removed when a sampling tube is connected to the luer connector. According to some embodiments, when a sampling tube is connected to the luer connector, the sealing of the sampling channel is automatically withdrawn. According to some embodiment, the seal is configured to seal around the sampling probe such that only exhaled air sampled from the inner diameter of the airway channel enters the sampling tube.

According to some embodiments, there is provided an airway system including a breath sampling tube; and any of the airway devices disclosed herein.

According to some embodiment, the system further comprises a ventilation and/or an anesthetics machine.

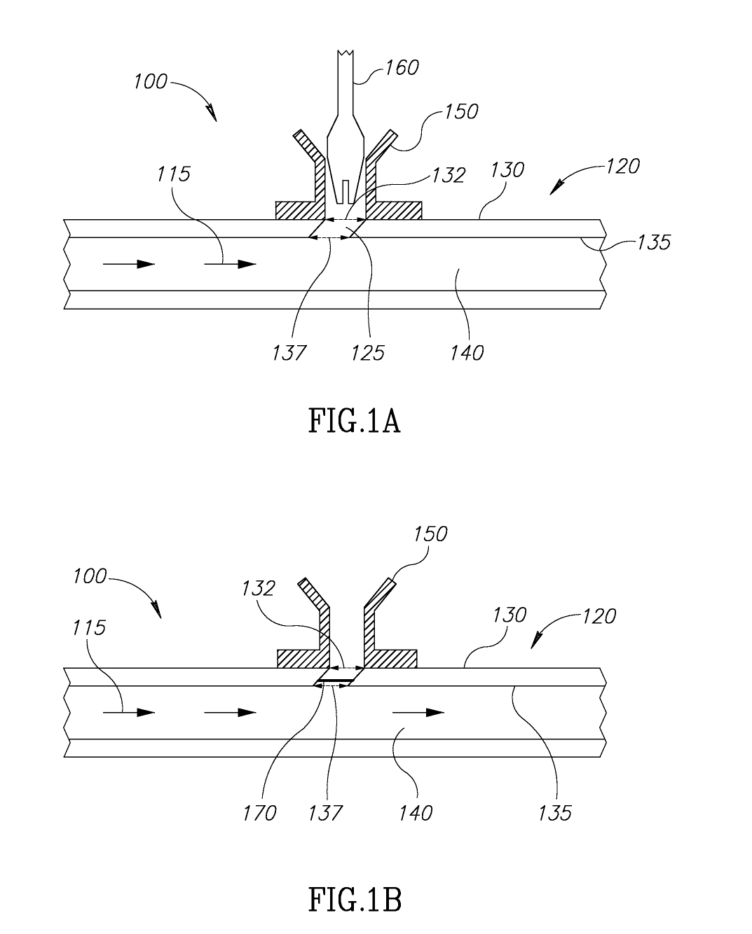

Reference is now made to FIGS. 1A and 1B which schematically illustrates an airway device 100 having a luer connector 150 positioned thereon, having or not having a sampling tube connected thereto, FIGS. 1A and 1B, respectively. Airway device 100 includes a shaft 120 having an outer wall 130 and an inner wall 135 defining an airway channel 140 configured to allow respiratory gasses (from the ventilation machine to the patient and vice versa) therein. Shaft 120 has a sampling channel 125 extending through inner wall 135 and outer wall 130. Sampling channel 125 is sloped relative to airway channel 140 such that the opening 137 in inner wall 135 is distal to, closer to the patient, as compared to opening 132 in outer wall 130. Such configuration serves to “assist” exhaled breath, flowing away from the patient (illustrated by arrow 115), to enter sampling channel 125 whereas respiratory gasses provided by a ventilation machine (not shown) and flowing in the direction to the patient is less likely to enter sampling channel 125. Luer connector 150 is located on outer wall 130 in fluid flow connection with sampling channel 125, thereby allowing gasses (exhaled breath) flowing in sampling channel 125 to enter a sampling tube 160 connected to luer connector 150, as shown in FIG. 1A. When no sampling tube is connected to luer connector 150 (as in FIG. 1B), a seal 170 is configured to seal of sampling channel 125, either at opening 137 of inner wall 135, as shown here, or at opening 132 of outer wall 130 (not shown) thereby avoiding leakage of respiratory gasses flowing in airway channel 140.

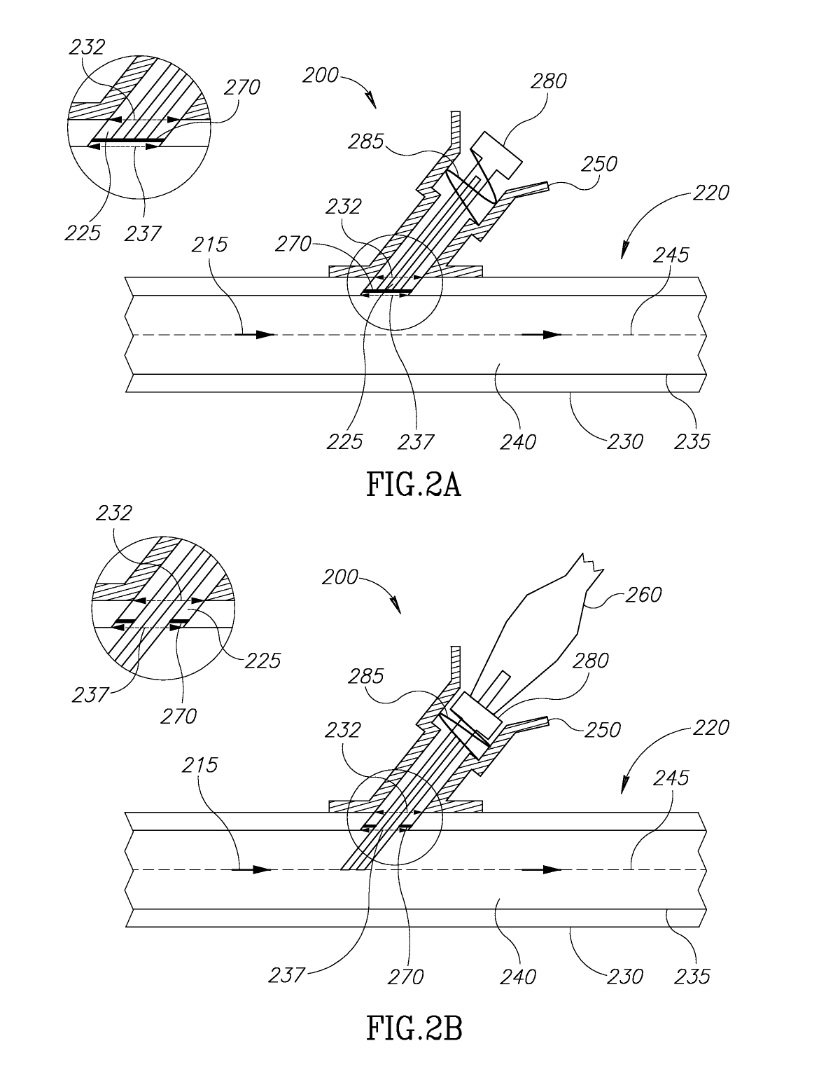

Reference is now made to FIGS. 2A and 2B which schematically illustrates an airway device 200 having a luer connector 250 positioned thereon, before and after connection of a sampling tube thereto, FIGS. 2A and 2B, respectively. Airway device 200 includes a shaft 220 having an outer wall 230 and an inner wall 235 defining an airway channel 240 configured to allow respiratory gasses (from the ventilation machine to the patient and vice versa) therein. Shaft 220 has a sampling channel 225 extending through inner wall 235 and outer wall 230. Sampling channel 225 is sloped relative to airway channel 240 such that the opening 237 in inner wall 235 is distal to the opening 232 in outer wall 230. Such configuration serves to “assist” exhaled breath (illustrated by arrow 215), flowing away from the patient, to enter sampling channel 225 whereas respiratory gasses provided by a ventilation machine (not shown) and flowing in the direction to the patient is less likely to enter sampling channel 225. Luer connector 250 is located on outer wall 230 above opening 232 in outer wall 230. Luer connector 250 includes a sampling probe 280 configured to sample breath from an inner diameter 245 of airway channel 240. Sampling probe 280 is connected to a spring 285 such that sampling probe 280 is normally retracted within luer connector 250 (FIG. 2A). When a sampling tube 260 connected to luer connector 250, as shown in FIG. 2B a force is applied on spring 285, pushing sampling probe 280 into airway channel 240 thereby facilitating sampling from inner diameter 245. Upon disconnection of sampling tube 260 from luer connector 250, sampling probe 280 returns to its retracted position within luer connector 250. Moreover, when no sampling tube is connected to luer connector 250 (FIG. 2A), a seal 270 is configured to seal of sampling channel 225, either at opening 237 of inner wall 235, as shown here, or at opening 232 of outer wall 230 (not shown) thereby avoiding leakage of respiratory gasses flowing in airway channel 240. When sampling tube 260 is connected to luer connector 250 (FIG. 2B), seal 270 is configured to seal around sampling probe 280, such that only exhaled breath sampled by sampling probe 280 enters sampling tube 260.

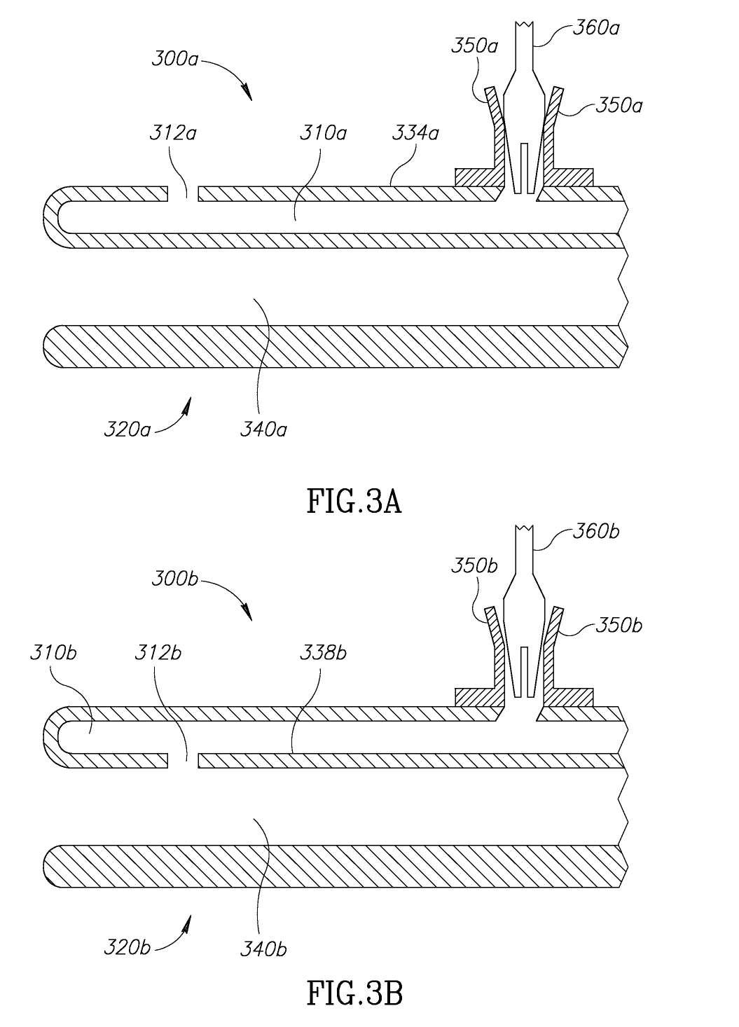

Reference is now made to FIG. 3A which schematically illustrates an airway device 300a having a luer connector 350a positioned thereon. Airway device 300a includes a shaft 320a having an airway channel 340a configured to allow respiratory gasses (from the ventilation machine to the patient and vice versa) therein. Shaft 320a has a sampling lumen 310a extending in and along shaft 320a, thereby forming a double lumen structure therewith. Sampling lumen 310a includes a notch 312a configured to allow gas to flow into the sampling lumen. Notch 312a is located in an external surface 334a of shaft 320a, such that respiratory gases (exhaled breath) may enter sampling lumen 310a directly from the patient (not shown) and further to sampling tube 360a connected to luer connector 350a. It is understood by one of ordinary skill in the art that this configuration requires that notch 312a is located on a part of shaft 320a configured to be entered into the patient's body when the airway device is in use, whereas luer connector 350a is configured to be connected to sampling tube 360a outside the patient's body and is positioned on part of shaft 320a remaining outside the patient's body when airway device 300a is in use.

Reference is now made to FIG. 3B which schematically illustrates an airway device 300b having a luer connector 350b positioned thereon. Airway device 300b includes a shaft 320b having an airway channel 340b configured to allow respiratory gasses (from the ventilation machine to the patient and vice versa) therein. Shaft 320b has a sampling lumen 310b extending in and along shaft 320b, thereby forming a double lumen structure therewith. Sampling lumen 310b includes a notch 312b configured to allow gas to flow into the sampling lumen. Notch 312b is located in an internal surface 338b of shaft 320b, such that respiratory gases (exhaled breath) may enter sampling lumen 310b from within airway channel 340b and further to sampling tube 360b connected to luer connector 350b. It is understood by one of ordinary skill in the art that notch 312b is configured to be located on a part of shaft 320b configured to be entered into the patient's body or on a part of shaft 320 being outside the patient's body. Luer connector 350b is configured to be connected to sampling tube 360b outside the patient's body and is therefore positioned the part of shaft 320b remaining outside the patient's body when the airway device is in use.

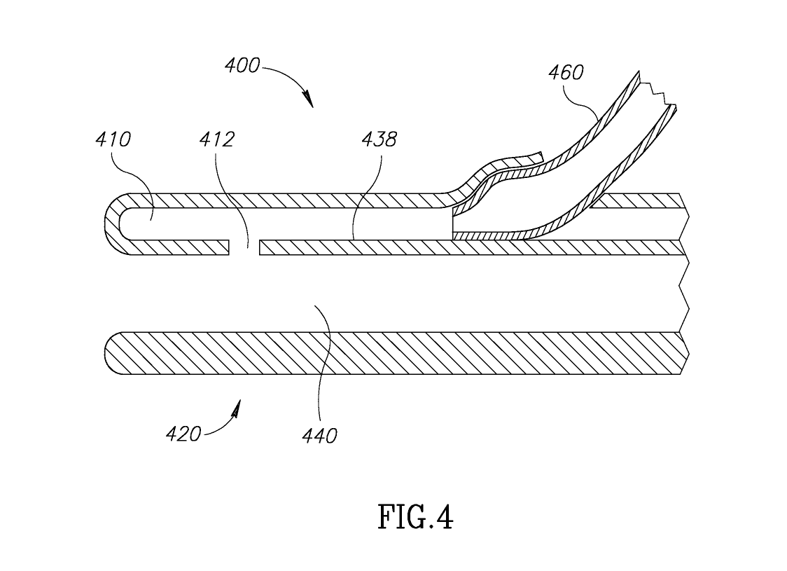

Reference is now made to FIG. 4 which schematically illustrates an airway device 400 having a sampling lumen 410 coextensive with as sampling tube 460. Airway device 400 includes a shaft 420 having an airway channel 440 configured to allow respiratory gasses (from the ventilation machine to the patient and vice versa) therein. Shaft 420 has a sampling lumen 410 extending in and along shaft 420, thereby forming a double lumen structure with airway channel 440. Sampling lumen 410 includes a notch 412 configured to allow gas to flow into the sampling lumen. Notch 412 is here located in an internal surface 438 of shaft 420, allowing respiratory gases to enter sampling lumen 410 from airway channel 440. Sampling lumen 410 is formed coextensive with sampling tube 460, which is thus an integral part of airway device 400, thereby allowing airway device 400 to be directly plugged in to a breath analyzer (not shown) through sampling tube 460.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising”, when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What is claimed is:

1. An airway device comprising:
a shaft having an airway channel configured to allow flow of respiratory gasses, said airway channel having an outer and an inner wall;
a sampling channel; and
a luer connector mounted on said outer wall such that respiratory gasses are allowed to flow from said airway channel through said sampling channel and said luer connector to a sampling tube connected to said luer connector.

2. The airway device of claim 1, wherein said sampling channel extends through said inner and outer walls of said airway channel.

3. The airway device of claim 1, wherein said sampling channel is sloped relative to the airway channel.

4. The airway device of claim 1, wherein said sampling channel is embedded in said outer wall of said airway channel.

5. The airway device of claim 4, wherein said sampling channel extends along at least a part of said airway channel.

6. The airway device of claim 4, further comprising a notch configured to allow gas to flow into said sampling channel.

7. The airway device of claim 6, wherein said notch is located in an outer surface of said shaft.

8. The airway device of claim 6, wherein said notch is located in an inner surface of said shaft.

9. The airway device of claim 6, wherein said notch is positioned in a distal end of said shaft inside a patient's body.

10. The airway device of claim 1, wherein said luer connector is mounted on a proximal end of said shaft externally to a patient's body.

11. The airway device of claim 1, wherein said luer connector comprises a sampling probe configured to sample gas from an inner diameter of said airway channel.

12. The airway device of claim 1, wherein said sampling probe is spring loaded.

13. The airway device of claim 1, wherein said sampling probe is normally retracted within said luer connector.

14. The airway device of claim 1, wherein said sampling probe enters into said airway channel when said sampling tube is connected to said luer connector.

15. The airway device of claim 1, wherein said luer connector further comprises a seal configured to prevent gasses flowing in said airway channel to exit through said luer connector when no sampling tube is connected thereto.

16. The airway device of claim 1, being an endotracheal tube, an endobroncheal tube or a tracheostomy tube.

17. The airway device of claim 1, configured to be connected to a ventilation, machine or an anesthetics machine.

18. An airway device comprising:
a shaft having an airway channel configured to allow flow of respiratory gasses;
a sampling channel extending along at least a part of said airway channel; and
a sampling tube coextensive with and permanently attached to said sampling channel.

19. An airway system comprising:
a breath sampling tube; and
an airway device comprising
a shaft, said shaft having an airway channel configured to allow gas flow therein, said airway channel having an outer and an inner wall;
a sampling channel; and
a luer connector mounted on said outer wall such that part of the gas flowing in said sampling channel is allowed to exit through said luer connector to said sampling tube connected to said luer connector.

20. The airway system of claim 19, further comprising a ventilation machine and/or an anesthetics machine.
